(19) United States
(12) Patent Application Publication
(10) Pub. No.: US 2018/0256136 A1
(43) Pub. Date: Sep. 13, 2018

(54) STOOL COLLECTION DEVICE AND STOOL SAMPLING DEVICE

(71) Applicant: Brian James MCCORMACK, By Kirkcaldy, Fife (GB)

(72) Inventor: Brian James MCCORMACK, By Kirkcaldy, Fife (GB)

(21) Appl. No.: 15/757,271

(22) PCT Filed: Sep. 5, 2016
(86) PCT No.: PCT/GB2016/052733

§ 371 Date: Mar. 2, 2018

(30) Foreign Application Priority Data
Sep. 3, 2015 (GB) 1515628.4
Feb. 16, 2016 (GB) 1602737.7

(51) Int. Cl. A61B 10/00

(52) U.S. Cl. CPC A61B 10/0038; A47K 17/00

(57) ABSTRACT

A stool collection device (10) comprising a rigid dissolvable fibre sheet adapted to fit above the water in a toilet pan.

The present invention relates to a stool collection device and a stool sampling device.

Currently when a patient receives a test for bowel cancer through the post no device is provided to aid the collection of the stool. If as an example the step-by-step instructions issued by the Scottish Bowel Screening Centre of NHS Scotland is consulted they list three methods to collect a sample. These include placing a large amount of tissue in a toilet bowl, catching the stool in your hand or a clean disposable container. Once deposited the stool sample has to be sampled and placed in a container for return to the testing centre. The methods provided by NHS Scotland are firstly difficult, secondly can lead to contamination of the sample thus providing a spurious result and thirdly in the case of the container create contaminated waste. In hospitals a cardboard bedpan is provided and sat in the toilet bowl. Whilst this provides a significantly more secure method for the sampling it still leaves the issue of disposing of the cardboard bedpan. This often entails taking the soiled bedpan through the hospital ward, which once again risks further contamination.

In the same kit as the advice leaflet on how to collect a stool sample is a stool sampler. This is a folded over piece of cardboard containing two sample points and a series of cardboard sticks for sampling. Once the stool sample has been deposited or collected the stick is used to sample the bowel motion in four different sites. The collected material spread over one window provided in the sampler. This is then repeated and sample spread on a second spot in the sampler using a fresh stick. The sampler is then folded together, held together by a tab ensuring the samples are covered and placed in receptacle to be sent back in the post. This has the disadvantage particularly with the paper method described above of ending up with contaminated samples as the stick can go through the paper. Furthermore by stabbing in four places this increases this chance substantially. Furthermore the collection and smearing of the sample is something that most users find unpleasant.

Previously proposed stool collection devices are described in WO2013/129930 and WO2009/129811 which both require mounting on the water closet.

The present invention seeks to ease the complexity of stool sampling.

Accordingly a first aspect of the present invention is directed to a stool collection device comprising a dissolvable sheet adapted to fit above the water in a toilet pan.

This provides the advantage that the stool can simply be provided onto the sheet that has been placed in the toilet bowl. This provides a platform upon which a sample can easily be removed from the stool. This has the added advantage that a user of a bowel cancer test kit is far more likely to use this device due to its simplicity and hygiene. Finally it has the advantage that the toilet when flushed will dissolve the sheet thus removing the contaminated material.

In a preferred embodiment the sheet has one or two holes in the centre part. This allows a user to hold the sheet and place it in the toilet pan above the water. Alternatively a cut can be provided in the sheet in order to enable a tab to be pushed out in a central portion of the sheet. This provides the advantage that the tab can be pushed out so as to protrude upwards enabling the placement of the sheet in the toilet pan. This ensures that users' fingers do not go near the water in the toilet pan. Obviously the pre-scored cut to provide the tab can have a variety of shapes such as a U-shape or a rectangle.

In a preferred embodiment a V- or U-shaped cut out is provided in one side of the sheet in order to enable the passage of urine if accidentally discharged without wetting the sheet.

The corners of the sheet are preferably curved to match the inside of the toilet pan. Furthermore the corners can have close to them serrations cut out in order to enable the sheet to conform better to the shape of the pan. Again these cut-outs can either be removed before use or pre-scored and detach upon use.

Advantageously the size of the sheet is designed to fit in an envelope, preferably a C5 envelope. This has the advantage that the stool collection device can be easily mailed in testing kits.

The sheet can comprise sprayed paper fibre, dissolvable paper, dissolvable cellulose, papier mâché, degradable pulp board, dissolvable fabric and sheets created from the spraying of paper pulp onto a wire mesh shape or combinations thereof.

Advantageously the thickness of the sheet is between 2 and 3 mm. This is such that it provides a level of rigidity while still enabling dissolution of the fibre material upon flushing of the lavatory.

The material from which the stool collection device is made is rigid however the level of stiffness is such as is necessary to perform its function. It may be necessary from a point of view of use and transport for the material to be bendable. The rigidity is sufficient that when in use the stool collection device will not deform such that the stool sample ends up in the water containing part of the toilet.

Preferably the sheet is constructed such that it has two halves which are mirror images or inverted mirror images of each other. In a preferred embodiment the join of the two halves has a pre-scored bendable axis. This provides the advantage that the stool collection device can be folded in two to fit in a postally sent collection pack.

In a preferred embodiment the sheet has four incisions cut, one in each corner. This gives the stool collection device an appearance of a Maltese style or German style cross.

This provides the advantage that the stool collection device can be compacted at the edge to create a bowl shaped container that fits into a lavatory for instance in a hospital thus providing a stable platform for significant sampling.

In a further embodiment the shape of the sheet is substantially circular or circular with two flattened edges with at least two incisions pre-scored or cut. Preferably the shape of the sheet is such that is rectangular with circular ends with at least two incisions. This provides the advantage that the stool collection device will fit in substantially most lavatories and can be easily folded in two and fit in a C5 envelope. The incisions can either be straight cuts or cut out segments. Advantageously there can be four incisions. The incisions are such that they cut out segments roughly at right angles to each other. Preferably at least two incisions are in the middle of each flattened edge with the pre-scored axis between them. Alternatively two incisions are in the middle of each curved end.

Preferably at least two incisions are equipped with joining means. This has the advantage that the when joined together the incisions are compressed from a bowl shape which is held. The joining means can be a hook like protrusion in the incision or a semi-circular protrusion pre-scored next to the incision and an accepting cut out next to the incision, preferably a hole. The hole preferably has a half moon shape. Preferably in the case of the semi-circular protrusion and the half-moon shaped hole, the half-moon shaped hole is approximately twice the size of the protrusion.

This provides the advantage that a shaped container is formed. Whist it can be used as such alternatively it can provide a dome shaped stool collection device that will fit in larger lavatories such as those found in the United States.

In a preferred embodiment the edge of the sampler can be provided with castellations. This provides the advantage particularly with larger lavatory bowls that the sampler grips the side of the bowl stopping the sampler touch the water in the pan.

Accordingly a second aspect of the present invention is directed to a stool sampling device comprising two sides joined together along one edge, the joined edge holds between the two sheets at least one stool stick, both walls having a bendable part close to the joined side such that two foldable flaps are created, which when folded back allow the stick to protrude.

This provides the advantage that a user can hold the joined side of the sampler and when the flaps are folded back allow the stick to take a sample of the bowel motion in a plurality of positions whilst protecting the user's hand from contamination.

In a preferred embodiment there are four sticks. This provides the advantage that the four samples required by for instance in the bowel cancer screening can prove be provided from a plurality of positions simply at one go. It will be appreciated that the number of sticks can be adjusted to the sampling process required be it one, two, three etc.

Preferably the edges of the side flaps have a cut securing tab or adhesive material on them such that when they are folded together they stick together holding the samples in place. Preferably before use the adhesive parts are protected by a film that is removed when it is proposed to use the sampler.

Preferably the sampler is made from cardboard material this provides simple cheap economic material. In a preferred embodiment the two sides are part a single folded sheet. Advantageously the stool sticks are held between the joined edge of the side closest to the fold.

Examples of stool collection devices and a stool sampler made in accordance with the present invention will now be described hereinbelow with reference to the accompanying drawings, in which:

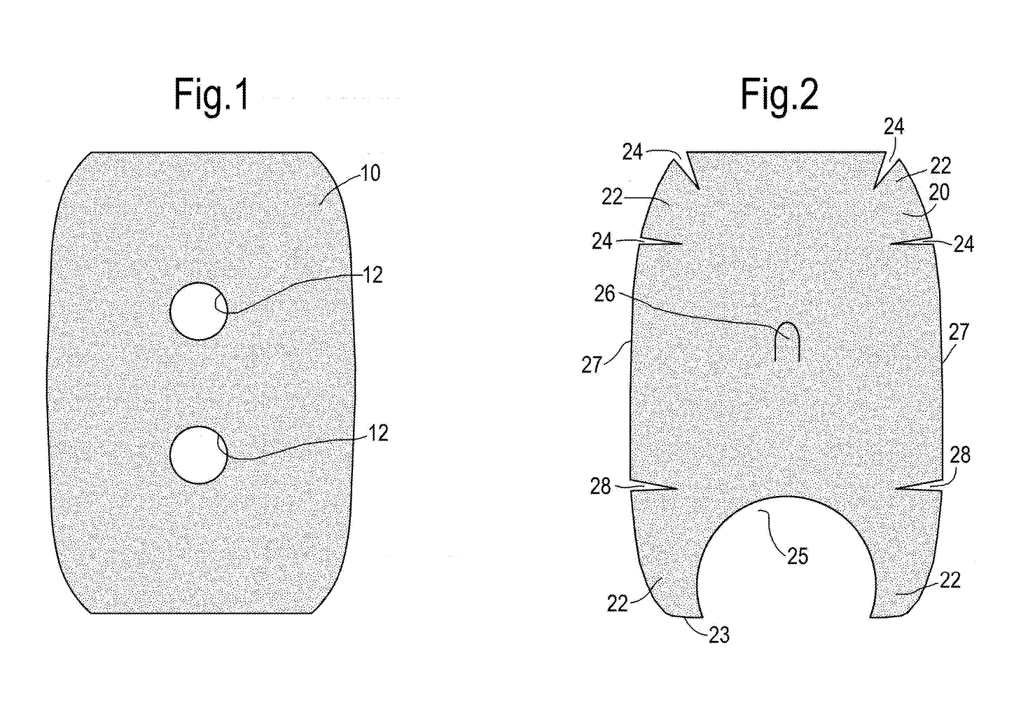

FIG. 1 shows a plan view of a first embodiment of the present invention;

FIG. 2 shows a plan view of a second embodiment of the present invention;

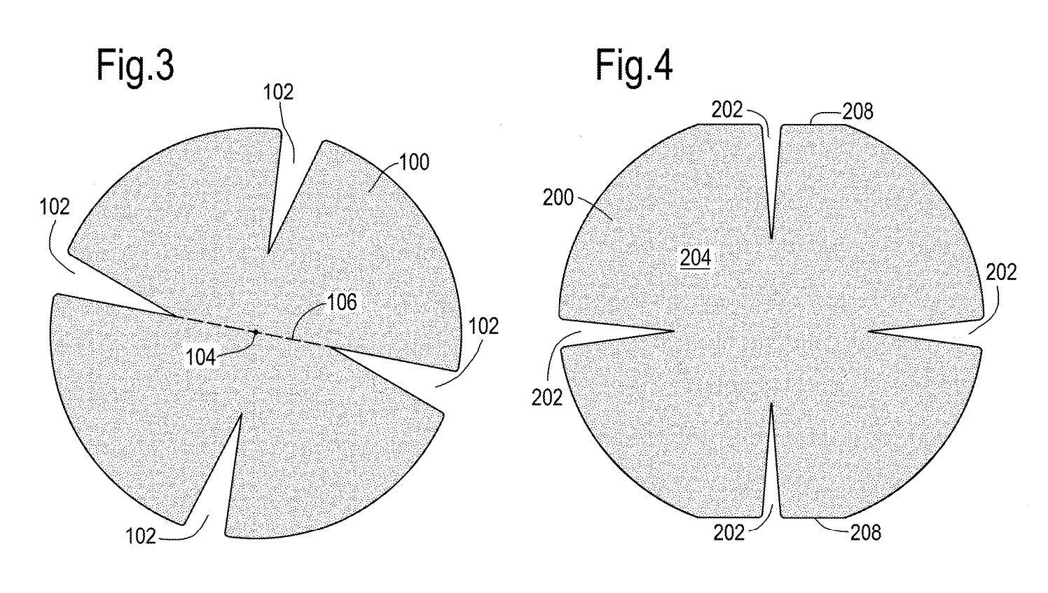

FIG. 3 shows a plan view of a third embodiment of the present invention;

FIG. 4 shows a plan view of a fourth embodiment of the present invention;

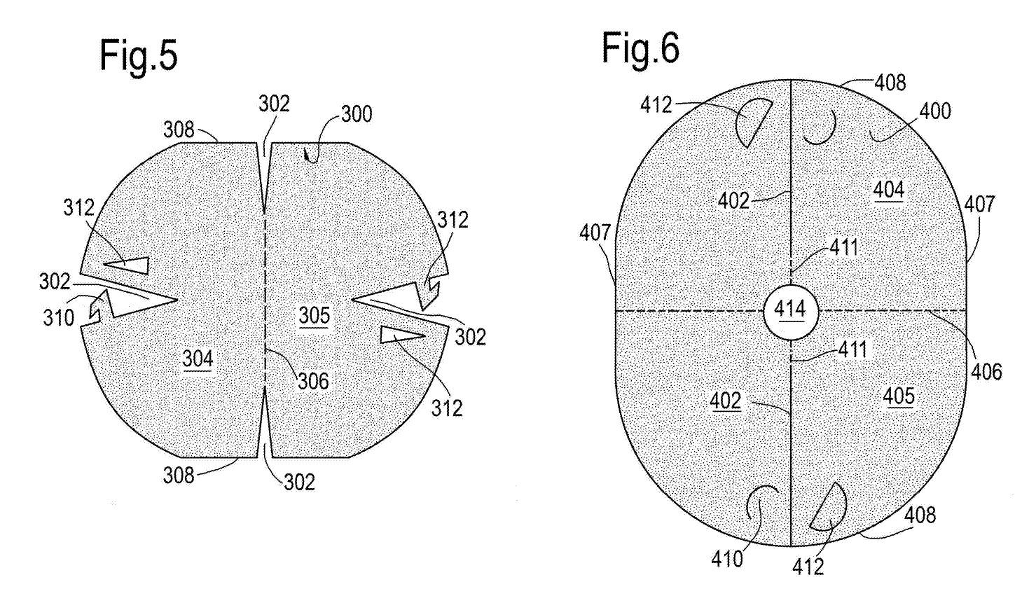

FIG. 5 shows a plan view of a fifth embodiment of the present invention;

FIG. 6 shows a plan view of a sixth embodiment of the present invention;

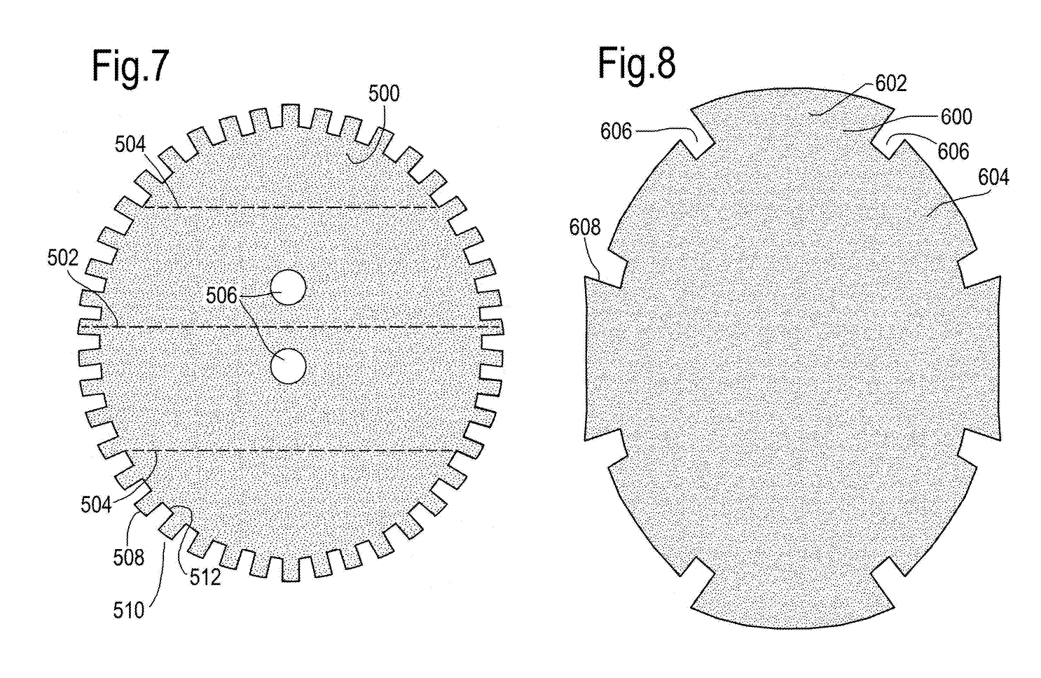

FIG. 7 shows a plan view of a seventh embodiment of the present invention;

FIG. 8 shows a plan view of an eighth embodiment of the present invention;

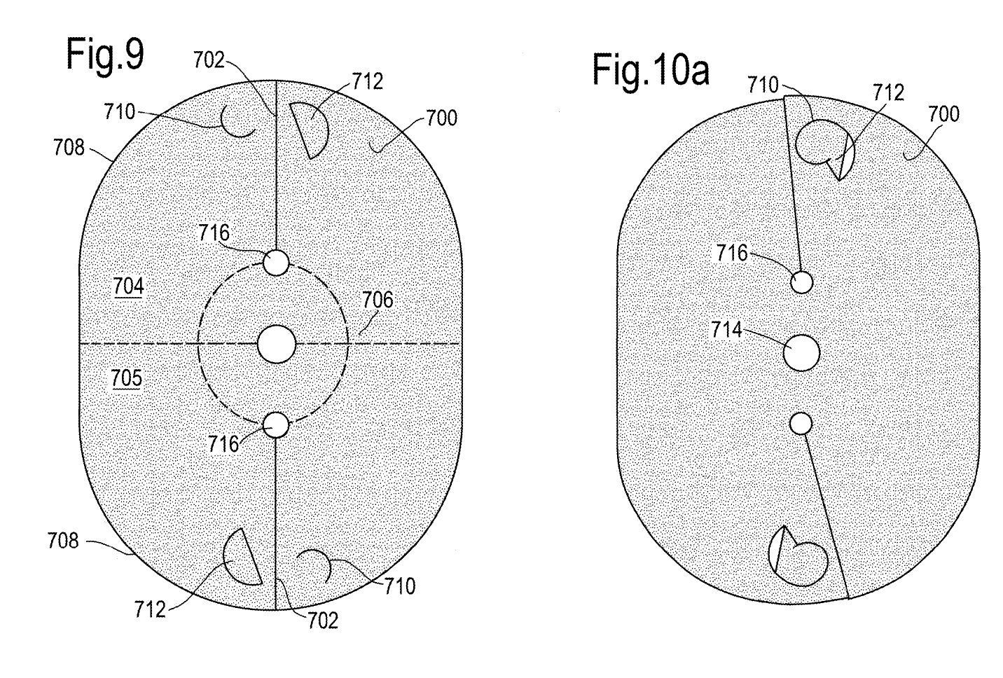

FIG. 9 shows a plan view of a ninth embodiment of the present invention;

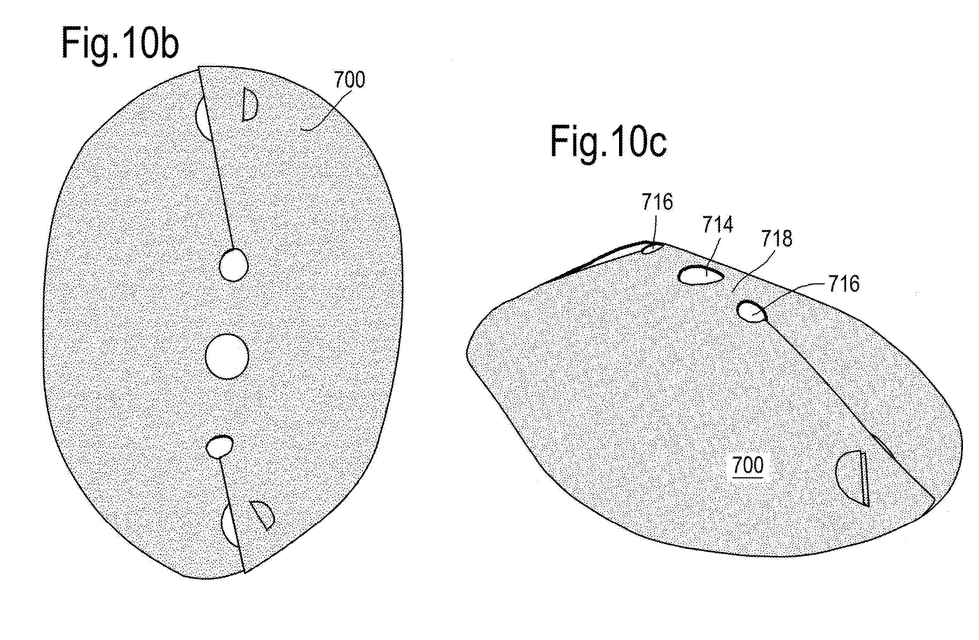

FIG. 10 shows views of the stool sampler in FIG. 9 assembled ready for use

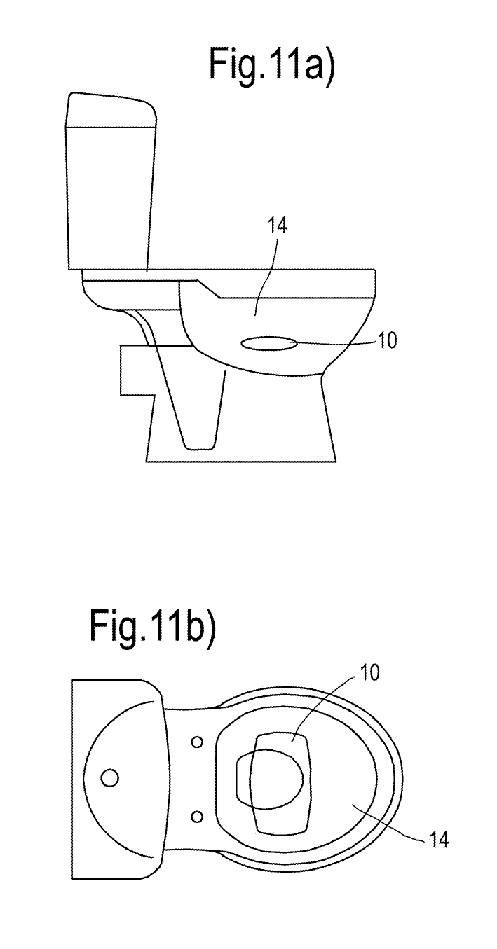

FIG. 11 shows a lavatory with the stool collection device of

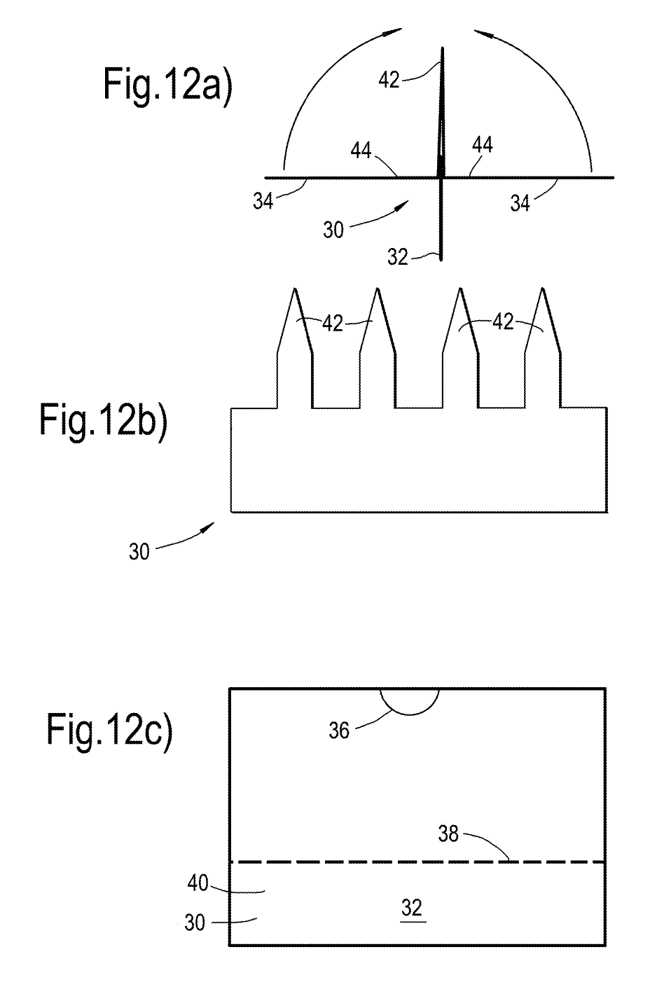

FIG. 1 installed in the toilet bowl; and FIG. 12 shows a stool sampler.

FIG. 1 shows a stool collection device 10 in plan view. The stool collection device 10 is a rectangular fibre sheet with the corners curved. This is designed to fit the majority of lavatory bowls. In this case the dimensions of the stool collection device are a width of 5¾ inches and a length of inches. Obviously this size could be amended depending upon the desired lavatory in which it is be used or alternatively based on the desired postal delivery method. In the middle of the sheet are two holes 12. These are designed to accept the thumb and index finger of a user such that the user can place the stool collection device at an appropriate position in the lavatory. The thickness of the sheet is preferably 2 to 3 mm with a weight of approximately 2 ounces. Obviously should a larger stool collection device be necessary these weights and thicknesses may need to vary.

FIG. 2 shows a stool collection device 20 in plan view. The stool collection device 20 is a rectangular fibre sheet with the corners curved 22. In the middle of one side 23 is a semi-circular cut out 25 for urination. The two corners 22 away from the cut out 25 each have serrations 24 cut in the sheet 20. One at the corner 22 and one each close thereto along the sides 27. Two further serration 28 are proved further along sides 27. The serrations ensure when in use the stool collection device 20 matches the curves of the toilet pan and thus fit in there in stable manner. In the middle of the sheet is a cut out tab 26 which when pushed upwards as the tab 26 allows the user can place the stool collection device at an appropriate position in the lavatory.

FIG. 3 shows a stool sampler 100 of substantially circular shape. The stool sampler 100 has four segments 102 cut out of its edge at roughly right angles. The cut-out segments 102 stretch a significant distance towards the centre 104 of the stool sampler 100. An axis 106 between two opposite cut-out segments 102 is pre-scored to enable the sampler 100 to be folded in two. This enables the sampler 100 to fit in an A5 envelope for example.

FIG. 4 shows a stool sampler 200. The sampler has a generally circular shape with two flattened edges 208, which are opposite each other. The sampler 200 has four segments 202 cut out of it at roughly right angles. Only three cut-out segments 202 are shown in the Figure. Two cut-out segments are approximately in the middle of the flattened edges 208. The two halves of the sampler 204 and 206 of mirror images. The cut-out segments 202 stretch sufficiently far into the sampler 200 such that when the edges of the sampler 200 are pushed together the sampler 200 has a bowl shape. In flat form the sampler 200 has a diameter of approximately an 8 inches diameter and when compressed to create the bowl shape this has a depth of approximately 1½ inches. This enables significant sampling and is designed to fit the bowl of a toilet.

FIG. 5 shows a stool sampler 300. The sampler has a generally circular shape with two flattened edges 308, which are opposite each other. The sampler 300 has four segments 302 cut out of it at roughly right angles. Two cut-out segments 302 are approximately in the middle of the flattened edges 308. An axis 306 between these two opposite cut-out segments 302 is pre-scored to enable the sampler 300 to be folded in two. The cut out 302 in each half 304 and 305 have a hook like protrusion 310 jutting into the cut out 302 from one side of the cut out 302. Next to the opposite side of the cut out 302 is found a triangular shaped hole 312 with the base of the triangle facing the axis 306. The two halves of the sampler 304 and 306 are inverted mirror images. The cut-out segments 302 stretch sufficiently far into the sampler 300 such that when the edges of the sampler 300 are pushed together the sampler 300 has a bowl shape. When compressed the hooks 310 fit into the holes 312 and hold the stool sampler 300 in the bowl form. In flat form the sampler 300 has a diameter of approximately a 8-9 inches diameter and when compressed to create the bowl shape this has a depth of approximately 1½ inches. This enables significant sampling and is designed to fit the bowl of a toilet.

FIG. 6 shows a stool sampler 400. The sampler has a rectangular circular shape with two circular ends 408, which are opposite each other. The sampler 400 has two incisions 402 which are pre-scored or cut in approximately the middle of each circular end 408 along one axis of the sampler 400. An axis 406 between these two opposite parallel sides 407 of the sampler is pre-scored to enable the sampler 400 to be folded in two. Alternatively or in addition the perpendicular axis 411 can be pre-scored and with the incisions 402 the sampler can be folded in two. The incisions 402 each have a hole 412 in a half moon shape and a semi-circular cut to create a protrusion 410 on either side near each circular edge 408 of the sampler 400. Considered from axis 406 the two halves of the sampler 404 and 405 are inverted mirror images. In use the incisions 402 stretch sufficiently far into the sampler 400 such that when the edges of the sampler 400 are pushed together the sampler 400 has a bowl or dome shape. When compressed the protrusions 410 fit into the holes 412 and hold the stool sampler 400 in the bowl or dome form. In the centre of the sampler is a hole 414 to position the stool sampler in the toilet. This also makes forming the bowl/dome shape easier. In flat form the sampler 400 has a diameter of approximately a 8-9 inches diameter and when compressed to create the bowl shape this has a depth of approximately 1½ inches. This enables significant sampling and is designed to fit the bowl of a toilet. This design is also preferable to use by trained staff in a hospital.

FIG. 7 shows a stool sampler 500. The sampler 500 has an oval/circular shape. The sampler 500 has three pre-scored parallel lines 502, 504 which divide the sampler into four roughly equal in width portions when folded. One of the pre-scored lines 502 is a central axis. On either side of the pre-scored central axis 502 are two holes to position the sampler 500. The edge 508 of the sampler 500 has a castellated form with protrusions 510 followed by pockets 512 of roughly equal length round the whole of the edge. This is designed such that in lavatories with larger bowls the castellations grip the side of the toilet pan. This is particularly true with dissolvable paper as the moisture from the toilet pan aids the gripping.

FIG. 8 shows a stool sampler 600. The sampler 600 has an oval/circular shape. The sampler 600 is a development of the sampler shown in FIG. 7. The edge 608 of the sampler 600 has a castellated form with protrusions 602, 604 followed by pockets 606. The pockets 606 cover a significant smaller section of the edge 608 compared to the protrusions 602, 604. The protrusions 602, 604 are of a different distance when measured from the centre of the sampler. Such that the protrusion 602 sticks out further than the protrusions 604. This is designed such that in lavatories with larger bowls the castellations grip the side of the toilet pan but the two sizes of castellations allow one sampler to fit more than one size of toilet bowl.

FIG. 9 shows a stool sampler 700. The sampler has a rectangular circular shape with two circular ends 708, which are opposite each other. The sampler 700 has two incisions 702 which are pre-scored or cut in approximately the middle of each circular end 708 along one axis of the sampler 700. The inner end of each incision 702 is equipped with a hole 716. The incisions 702 each have a hole 712 in a half moon shape and a semi-circular cut to create a protrusion 710 on either side near each circular edge 708 of the sampler 700. Considered from axis 706 the two halves of the sampler 704 and 705 are inverted mirror images. In use the incisions 702 stretch sufficiently far into the sampler 700 such that when the edges of the sampler 700 are pushed together the sampler 700 has a bowl or dome shape. When compressed the protrusions 710 fit into the holes 712 and hold the stool sampler 700 in the bowl or dome form. In the centre of the sampler is a hole 714 to position the stool sampler in the toilet. This also makes forming the bowl/dome shape easier

FIG. 10 shows the sampler 700 assembled for use. FIG. 10a shows the inside of the dome/bowl with each incision 702 pushed together such that the edges of the incision 702 overlap. The edges of the incisions 702 are held together by the protrusion 712 being pushed out and held in the hole 712 with one end jutting against the outer corner of the half-moon hole 712. The pushing together of the edges of the incisions 702 is aided by the holes 714 which allows each side to slide easily over the other. FIG. 10b shows the other side of the sampler 700 which shows the relative smoothness of the joint. FIG. 10c shows a perspective view of the sampler 700 prepare for use. As is clear a dome like or bowl like structure is created with a top dome edge 718 between the holes 716 with the hole 714 in the middle for sighting of the sampler 700.

FIG. 11 shows a diagrammatic view of a water closet lavatory. The lavatory has a bowl 14. In the bowl 14 is placed the stool collection device 10. As can be seen from the Figure the stool collection device fits in the lavatory so that it is above where water would be expected and rests on the sides of the bowl 14 as shown in FIG. 11b.

In use the user would place the stool collection device into the lavatory bowl 14 using his thumb and forefinger to hold the device and position it at the appropriate point. The user would then deposit their stool. The stool would then be sampled using the device provided. Once this sample has been taken the water closet toilet would be operated with the water flushing the stool and the device away due to the action of the water. The stool device collection device would dissolve or disperse upon contact with the water through wetting. In order to achieve this it may be necessary to flush the lavatory more than once.

FIG. 12 shows a bowel motion sampler. The sampler 30 comprises a sheet of card folded back upon itself to create a rectangle with two walls 34. The fold 32 occurs along edge 32. On the edge furthest away from the fold 32 on one of the walls 34 there is a cut out 36 provided to secure the other wall 32 thereto. Alternatively the walls 36 could be joined by adhesive provided along that edge upon completion. Each wall 36 has a pre-scored bendable joint 38 running from one side wall to the other paralleling the edge 32. In the part 40 between the two walls 34 and between the fold 38 and the edge 32 are mounted four sampling sticks 42. The walls 34 in the area 40 are glued together to hold the sticks 42 in place. When the walls 34 are bent back along joint 38 they expose the sticks 42 and create two flaps 44.

Therefore in use the two flaps 44 pulled back to expose the sticks 42. The user then grips portion 40 and samples the bowel motion in four places. The flaps 44 prevent the fingers of the user being contaminated with the bowel motion. Once sampling is complete the flaps 44 folded back to create a flat envelope which is then secured by tab 36 or adhesive. The sampler 30 can then be sent to the screening service.

A stool sampler according to FIG. 5 made with 14 point Smartsolve dissolvable paper was tested in an Armitage Shanks Sandringham Classic toilet. The toilet has a flush of approximately 6 litres. A stool sample simulation comprising 11b of icing sugar was placed in the sampler. The stool sampler and simulated sample were both passed through the toilet pan on one flush.

1. A stool collection device comprising a dissolvable sheet adapted to fit above the water in a toilet pan.

2. A stool collection device according to claim 1, in which the sheet has one or two holes in the centre part.

3. A stool collection device according to claim 1, in which the sheet has a cut in the sheet in order to enable a tab to be pushed out in a central portion of the sheet.

4. A stool collection device according to any preceding claim 1, in which the sheet has a V- or U-shaped cut out is provided in one side of the sheet.

5. A stool collection device according to claim 1, in which the corners of the sheet are curved.

6. A stool collection device according to claim 1, in which the corners can have close to them serrations cut out in order to enable the sheet to shape.

7. A stool collection device according to claim 1, in which the sheet is designed to fit in an envelope, preferably a C5 envelope.

8. A stool collection device according to claim 1, in which the sheet comprises sprayed paper fibre, dissolvable paper, dissolvable cellulose, papier mâché, degradable pulp board, dissolvable fabric and sheets created from the spraying of paper pulp onto a wire mesh shape or combinations thereof.

9. A stool collection device according to claim 1, in which the thickness of the sheet is between 2 and 3 mm.

10. A stool collection device according to claim 1, in which the stool sampler is constructed such that it has two halves which are mirror images or inverted mirror images of each other.

11. A stool collection device according to claim 10, in which the join of the two halves has a pre-scored bendable axis.

12. A stool collection device according to claim 1, in which the stool sampler has four incisions cut, one in each corner.

13. A stool collection device according to claim 1, in which the shape of the sampler is substantially circular with four incisions.

14. A stool collection device according to claim 1, in which the shape of the sheet is substantially circular or circular with two flattened edges with at least two incisions pre-scored or cut.

15. A stool collection device according to claim 1, in which the shape of the sheet is rectangular with circular ends with at least two incisions.

16. A stool collection device according to claim 14, in which incisions can either be straight cuts or cut out segments.

17. A stool collection device according to claim 14 any one of claims 14 to 16, in which there are four incisions.

18. A stool collection device according to claim 17, in which the incisions are such that they cut out segments roughly at right angles to each other.

19. A stool collection device according to claim 14, in which at least two incisions are in the middle of each flattened edge with the pre-scored axis between them.

20. A stool collection device according to claim 15, in which two incisions are in the middle of each curved end.

21. A stool collection device according to claim 14, in which at least two incisions are equipped with joining means.

22. A stool collection device according to claim 21, in which the joining means are a hook like protrusion in the incision or a semi-circular protrusion pre-scored next to the incision and an accepting cut out next to the incision, preferably a hole.

23. A stool collection device according to claim 22, in which the hole has a half moon shape.

24. A stool collection device according to claim 23, in which the half-moon shaped hole is approximately twice the size of the protrusion.

25. A stool collection device according to claim 1, in which the edge of the sampler comprises castellations.
